(19) United States
(12) Patent Application Publication
(10) Pub. No.: US 2016/0287393 A1
(43) Pub. Date: Oct. 6, 2016

(54) MULTIPOINT STAMPING MOULD

(71) Applicant: UNIVERSITI MALAYA, Kuala Lumpur (MY)

(72) Inventors: Zainal Ariff Bin Abdul Rahman, KUALA LUMPUR (MY); Alwin Kumar Rathinam, KUALA LUMPUR (MY); Yuwaraj Kumar A/L Balakrishnan, KUALA LUMPUR (MY); Su Tung Tan, KUALA LUMPUR (MY)

(21) Appl. No.: 15/100,323

(22) PCT Filed: Apr. 21, 2014
(86) PCT No.: PCT/MY14/00063

§ 371 Date: May 30, 2016

(30) Foreign Application Priority Data
Apr. 30, 2013 (MY) PI 2013700702

(51) Int. Cl. A61F 2/30; B21D 22/02; A61B 17/88

(52) U.S. Cl. CPC A61F 2/30942; A61B 17/8863; B21D 22/02; A61F 2002/30957

(57) ABSTRACT

This invention relates to a multipoint stamping mould for forming a customised implant mould surface comprising a primary stamping unit (100) having a plurality of rods (101) positioned adjacent to and in contact with one another, each rod (101) having a rough surface along its longitudinal axis and a mould surface defining end (103), wherein the rods (101) are slidable about the longitudinal axis to thereby form a customised implant mould surface by the mould surface defining ends (103) of the rods, according to a predetermined implant surface of the customised implant; and a clamping means (105) for locking the plurality of rods (101) into a position that defines the mould surface. Preferably, the multipoint stamping mould further comprises a secondary stamping unit (200) positioned opposite and corresponding to the primary stamping unit (100).

### FIELD OF INVENTION

The present invention relates to a multipoint stamping mould. In more particular, the invention provides an adjustable and changeable multipoint stamping mould capable of forming a highly accurate customised implant mould surface.

### BACKGROUND OF THE INVENTION

An implant is manufactured to replace a missing biological structure, support a damaged biological structure, or enhance an existing biological structure. Surgical implants are man-made devices, in contrast to a transplant, which is containing biomedical tissue. The surface of the implant that is in contact to a body might be made of a biomedical material such as titanium, silicone or other biocompatible materials depending on what is the most applicable.

Surgical implants are generally customized to a patient's need. Surgeons conventionally use time-consuming manual process to design an estimated bone replacement by viewing and interpreting X-ray or computerised tomography scans. Changes of the produced implants before surgery are mostly needed and there will be potential inaccuracies in implant thickness, shape and curvature. This conventional process obviously requires lengthy time. Moreover, design of patient's implant by mere observation through medical images is highly susceptible to inaccurate implants, which have potential risks to the patient. Surgery might need to be halted to address any ill-fitting areas on the implant.

There are some patented technologies over the prior arts relating to an apparatus to produce a mould. As disclosed in a U.S. Pat. No. 5,931,216, an adjustable casting mould having a mould frame with a pair of stationary facing side walls and a pair of facing end walls, wherein one of the end walls can be displaced and a coolant chamber is assigned to each side wall and end wall. However, the mould frame merely has at least one displaceable wall and the shaping of a cast material is merely effected by the middle parts of the end and side walls.

A stamping machine composed of a frame supporting a movable carriage is disclosed in U.S. Pat. No. 4,535,617(A). The carriage movement is controlled by two barrier walls and a material is held by a clamp during the punching process.

In another U.S. Patent No. US005330343A, a device capable of forming a mould surface is also disclosed. The device is utilized by adjusting longitudinal positions of rods through a digitally controlled tool. The rods are helically threaded and they rotate to move longitudinally. The drawback of this prior art is that the device requires complex and lengthy computerized interpretation of mathematical functions to control the movement and mechanism of the rods.

A need therefore exists to develop a mould for producing varying and changeable mould surfaces that is efficient for stamping and pressing processes of customised surgical implants.

### SUMMARY OF INVENTION

The primary object of the present invention is to produce an adjustable and changeable mould for fabrication of customised surgical implants. The mould is adjustable to varying mould surfaces by longitudinal configurations of a plurality of laterally adjacent rods.

Another object of the present invention is to produce a mould that forms or changes into a desired mould surface in an extremely fast speed, with accurate configuration and is relatively cost effective. The mould is reusable and can be established with exact curvature of pre-injury bone structures within days.

At least one of the proceeding objects is met, in whole or in part, by the present invention, in which the preferred embodiment of the present invention describes a multipoint stamping mould for forming a customised implant mould surface comprising a primary stamping unit (100) having a plurality of rods (101) positioned adjacent to and in contact with one another, each rod (101) having a rough surface along its longitudinal axis and a mould surface defining end (103), wherein the rods (101) are slidable about the longitudinal axis to thereby form a customised implant mould surface by the mould surface defining ends (103) of the rods, according to a predetermined implant surface of the customised implant; and a clamping means (105) for locking the plurality of rods (101) into a position that defines the mould surface.

One of the preferred embodiment of the present invention discloses that the mould further comprising further comprising a secondary stamping unit (200) positioned opposite the primary stamping unit (100), the secondary stamping unit (100) comprising a plurality of rods (201) that are positioned adjacent to and in contact with one another and substantially parallel to the rods (101) in the primary stamping unit (100), each rod (201) has the rough surface along its longitudinal axis and a mould surface defining end (203) corresponding to the mould surface defining end (103) of the secondary stamping unit (100); and a clamping means (205) for locking the plurality of rods (201) into a position that defines the mould surface.

Another preferred embodiment of the present invention discloses that the rough surface of each rod is formed by a plurality of protuberances, annular ridges or a combination thereof. Each rod (101, 201) is frictionally engaged with the rough surface along its longitudinal axis.

Yet another embodiment of the present invention, the rough surface of each rod is made of an elastomeric material.

Another embodiment of the present invention discloses that the rods (101, 201) are made of biocompatible material, stainless steel or reinforced plastic.

Still another embodiment of the present invention describes that each rod (101, 201) is connected to a computer-controlled actuator at its another end (109, 209).

A further embodiment of the present invention discloses that the customised implant mould surface is used for forming a customised titanium steel plate.

The present preferred embodiments of the invention consists of novel features and a combination of parts hereinafter fully described and illustrated in the accompanying drawings and particularly pointed out in the appended claims; it being understood that various changes in the details may be effected by those skilled in the arts but without departing from the scope of the invention or sacrificing any of the advantages of the present invention.

### BRIEF DESCRIPTION OF THE DRAWINGS

For the purpose of facilitating an understanding of the invention, there is illustrated in the accompanying drawing the preferred embodiments from an inspection of which when considered in connection with the following description, the invention, its construction and operation and many of its advantages would be readily understood and appreciated.

FIG. 1 shows a front view of a mould as described in accordance to one of the preferred embodiments of the present invention.

FIG. 2 shows a perspective view of a mould having a primary and secondary stamping unit (100, 200) as described in accordance to another preferred embodiment of the present invention.

FIG. 3 shows a front view of a mould having a primary stamping unit (100) and a secondary stamping unit (200) in accordance to the one of the embodiments of the present invention.

### DETAILED DESCRIPTION OF THE INVENTION

Hereinafter, the invention shall be described according to the preferred embodiments of the present invention and by referring to the accompanying description and drawings. However, it is to be understood that limiting the description to the preferred embodiments of the invention and to the drawings is merely to facilitate discussion of the present invention and it is envisioned that those skilled in the art may devise various modifications without departing from the scope of the appended claim.

The present invention relates to a multipoint stamping mould. In more particular, the invention provides an adjustable and changeable multipoint stamping mould capable of forming a highly accurate customised implant mould surface.

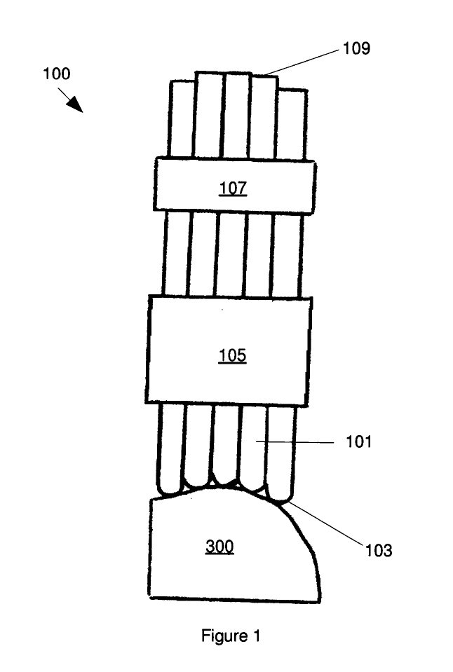

The present invention discloses a new approach for a multipoint stamping mould for forming a customised implant mould surface. The multipoint stamping mould of the present invention can be having either single or double stamping units. Referring to FIG. 1, the mould comprises a single, or a primary stamping unit (100) having a plurality of rods (101) positioned adjacent to and in contact with one another, each rod (101) having a rough surface along its longitudinal axis and a mould surface defining end (103), wherein the rods (101) are slidable about the longitudinal axis to thereby form a customised implant mould surface by the mould surface defining ends (103) of the rods, according to a predetermined implant surface of the customised implant; and a clamping means (105) for locking the plurality of rods (101) into a position that defines the mould surface.

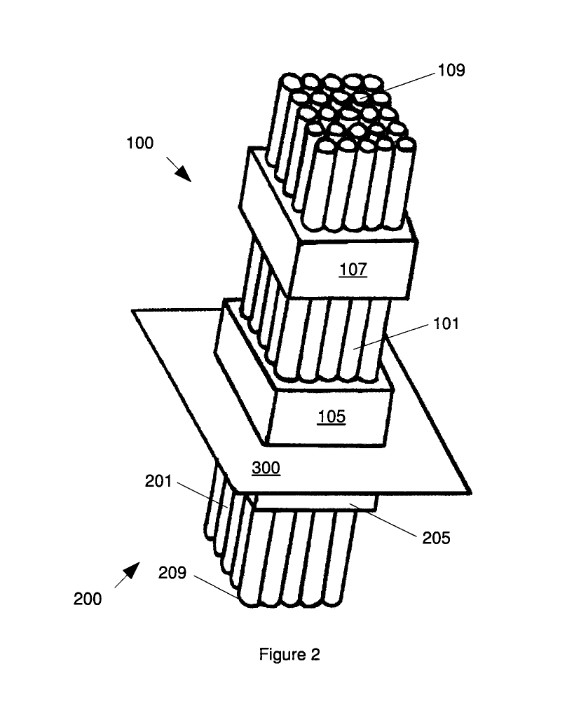

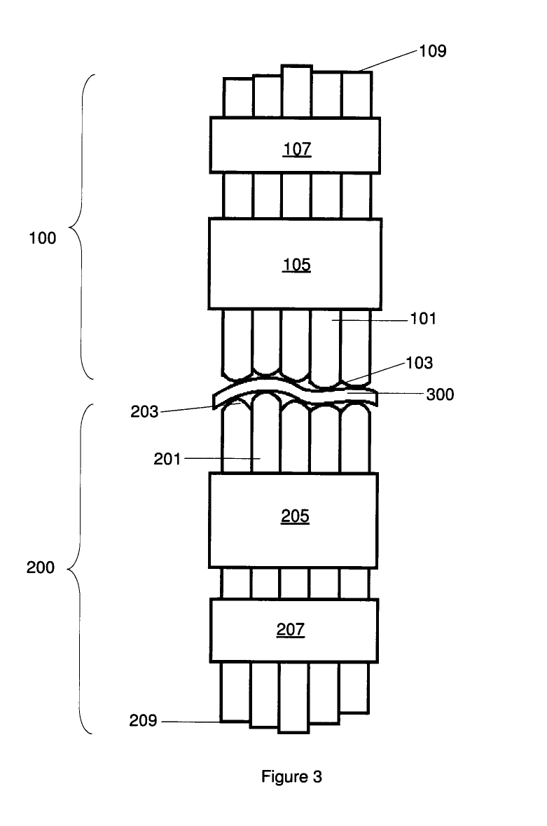

Now referring to FIGS. 2 and 3, the multipoint stamping mould further comprises a secondary stamping unit (200) positioned opposite the primary stamping unit (100), wherein the secondary stamping unit (200) comprises a plurality of rods (201) that are positioned adjacent to and in contact with one another and substantially parallel to the rods (101) in the primary stamping unit (100), each rod (201) has the rough surface along its longitudinal axis and having a mould surface defining end (203) corresponding to the mould surface defining end of the primary stamping unit (100); and a clamping means (205) for locking the plurality of rods (201) into a position that defines the mould surface.

According to a preferred embodiment of the present invention, the mould surface defines a surface of a customised implant or surgical instrumentation. For patients with missing or impaired bone structure, the customised implant can be used to replace the missing or impaired structure by aligning the implant with the patient's available anatomy, adjusting and smoothing out the shape and edges of the implant to match contours and thickness of the patient's available anatomy. High precision customised implant mould surface is essential for acceptance of tissues as well as to provide a pleasing appearance to the patient. The rods are preferred to be elongated and cylindrical and each rod having rounded ends to delineate an organically even surface of the implant. Additionally, the rounded end of each rod makes configuration of the organically complicated mould surfaces possible and accurate.

According to another preferred embodiment of the present invention, the rods (101, 201) are preferred to be in a specific diameter range depending on its applications. More preferably, the diameter of the rods (101, 201) is small for high dimensional precision of implant mould surfaces. Alternatively, larger rod diameter can be used for implants with less curvature.

In yet another embodiment of the present invention, the rough surface of each rod (101, 201) is formed by a plurality of protuberances, annular ridges or a combination thereof. The protuberances can be semi-spherical, triangular, square or criss-crossed surface. The protuberances help to frictionally retain the rods (101, 201) while permitting the rods (101, 201) to slide in contact with the laterally adjacent rods (101, 201). The plurality of rods is generally in a bundle. The rough surfaces along the longitudinal axis of the rods (101, 201) help to prevent slippage of the rods (101, 201) during the moulding process, especially rods (101, 201) in the centre of the bundle.

Based on the preferred embodiment of the present invention, the rough surface of each rod (101, 201) is preferred to be made of an elastomeric material such as rubber. The elastomeric material is preferred due to its high friction, flexible and compressible properties. The rods (101, 201) are held closely to one another when the clamping means (105, 205) tighten to secure the rods (101, 201), causing the elastomeric material between the rods (101, 201) to be pressed. Therefore, the rods (101, 201) in the centre of the bundle can be properly secured by the frictionally engaged rough surfaces.

Still another embodiment of the present invention discloses that the rods (101, 201) are made of biocompatible material, stainless steel or reinforced plastic. The rods (101, 201) are preferred to be made of high strength and corrosion resistance material. Most preferably, the rods (101, 201) are made of stainless steel.

Based on the embodiments of the present invention, the clamping means (105, 205) locks the rods (101, 201) firmly into position when the mould surface is configured. Bending or horizontal distortion of the rods (101, 201) can be prevented by the clamping means (105, 205) because the rods (101, 201) are secured by restricting horizontally movement of the rods (101, 201) when subjected to force, vibration or impact. Therefore, mechanical stability can be ensured during the moulding process.

In a further embodiment of the present invention, each rod (101, 201) in both the primary stamping unit (100) and secondary stamping unit (200) is connected to a computer-controlled actuator at its another end (109, 209), which is the non- mould surface defining end. The computer-controlled actuator is used for longitudinal movement of the rods (101, 201) to delineate the customised implant mould surface. The computer-controlled actuator can be a mechanical actuator, hydraulic actuator, pneumatic actuator, piezoelectric actuator or the combination thereof. It should be understood that any actuator can be used as long as they provide fine and repeatable position resolution as well as permit the rods (101, 201) to be fixed in position during the moulding process.

A further embodiment of the present invention discloses that the customised implant mould surface is used for forming a customised implant made from a moulding material (300). Preferably, the moulding material (300) is a titanium steel plate due to its durability as well as flexibility to form by pressing or stamping. The moulding material (300) is preferred to be positioned between the mould surface defining ends of the first and secondary stamping unit (100, 200). Pressing of both units (100, 200) towards one another can cause the moulding material (300) to take a shape and curvature of the mould surfaces, subsequently forming the customised implant.

The mould as described in the preferred embodiment of the present invention allows an implant or surgical instrumentation to be produced by moulding processes such as cold pressing, hot pressing or stamping directly on the configured mould surface. After the moulding process, the rods (101, 201) can be reconfigured to produce a new mould surface. A new mould surface configuration is preferred to be created through a control system or directly from a computing device, where the customised mould can be designed for better fit, accuracy and likelihood of long-term acceptance. The computer-controlled actuators are configured to move the rods (101, 201) into position according to the new mould design. Even more, movement of the rods (101, 201) can be adjusted manually by arranging the rods (101, 201) to the desired mould surface and securing the rods (101, 201) with the clamping means. Additional clamping means (107, 207) can also be equipped to the stamping units (100, 200) to facilitate the movement or adjustment of the rods (101, 201).

Moulding surface can be configured in the computing device for more precise changes and more refine edges in order to assure a comfortable fit for the patient. Customised implants allow patient's own bone structure to be preserved as much as possible and repair a damaged area with smooth edges that facilitate soft-tissue acceptance of the implant, as well as provide a pleasing appearance for the patient.

While the invention has been disclosed in connection with the preferred embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

1.-7. (canceled)

8. A multipoint stamping mould for forming a customised implant mould surface, comprising:
a first stamping unit having a first plurality of rods positioned adjacent to and in contact with one another;
wherein each rod of the first plurality of rods has a rough surface along its longitudinal axis and a first mould surface defining end;
wherein the rods of the first plurality of rods are slidable about the longitudinal axis to form a customised implant mould surface by the first mould surface defining end of the rods, according to a predetermined implant surface of the customised implant; and
a first clamping device for locking the first plurality of rods into a position that defines the mould surface.

9. The mould according to claim 8, further comprising:
a second stamping unit positioned opposite the first stamping unit, comprising:
a second plurality of rods that are positioned adjacent to and in contact with one another and substantially parallel to the first plurality of rods in the first stamping unit;
wherein each rod of the second plurality of rods has a rough surface along its longitudinal axis and a second mould surface defining end corresponding to the first mould surface defining end of the first stamping unit; and
a second clamping device for locking the second plurality of rods into a position that defines the mould surface.

10. The mould according to claim 9, wherein the rough surface of each rod of the first and second plurality of rods is formed by a plurality of protuberances, annular ridges or a combination thereof.

11. The mould according to claim 9, wherein the rough surface of each rod is made of an elastomeric material.

12. The mould according to claim 9, wherein the rods of the first and second plurality of rods are made of a biocompatible material, stainless steel or reinforced plastic.

13. The mould according to claim 9, wherein each rod of the first and second plurality of rods is connected to a computer-controlled actuator at another end.

14. The mould according to claim 9, wherein the customised implant mould surface is used for forming a customised titanium steel plate.
